(19) United States
(12) Patent Application Publication
(10) Pub. No.: US 2011/0166660 A1
(43) Pub. Date: Jul. 7, 2011

(54) IMPLANT WITH SPIRAL ANCHOR

(75) Inventor: Lawton Laurence, West Chester, PA (US)

(73) Assignee: SYNTHES USA, LLC, West Chester, PA (US)

(21) Appl. No.: 13/061,372

(22) PCT Filed: Sep. 2, 2009
(86) PCT No.: PCT/US2009/055747

§ 371 Date: Feb. 28, 2011

Related U.S. Application Data
(60) Provisional application No. 61/093,552, filed on Sep. 2, 2008.

(51) Int. Cl. A61F 2/44

(52) U.S. Cl. 623/17.16

(57) ABSTRACT

An intervertebral implant (100) for insertion into an intervertebral disc space between adjacent vertebral bodies. The implant includes an intervertebral spacer (105) and a spiral anchor (110) for securely coupling to both the intervertebral spacer and to at least one of the adjacent vertebral bodies. The at least one spiral anchor is configured to partially embed within a portion of the vertebral body.

### CROSS-REFERENCE TO RELATED APPLICATIONS

This application claims the benefit of U.S. Provisional Application No. 61/093,552, filed on Sep. 2, 2008, entitled “FUSION IMPLANT WITH SPIRAL ANCHOR,” the contents of which is incorporated in its entirety by reference herein.

### BACKGROUND OF THE INVENTION

Intervertebral implants for spinal fusion that are inserted into an intervertebral disc space between adjacent vertebral bodies and which allow growth of bone from adjacent vertebral bodies through the upper and lower surfaces of the implant are generally known. Such implants may be provided with a lordotic taper to enable a surgeon to recreate a lordotic curvature to the motion segment. In order to create an environment for fusion, fixation hardware is applied to the spinal segment to limit the relative motion between the vertebral bodies. As a result, interbody implants that feature a screw thread form connected to a central body have been developed, such as cylindrically threaded spacers. These devices are typically hollow and allow bone growth through fenestrations in the device.

Dynamic total disc replacement implants are also know and are inserted into a disc space to maintain motion between adjacent vertebrae. The disc replacement implant typically includes endplates that are secured to the vertebral bodies and a motion element therebetween that may be comprised of a ball and socket-type joint, a flexible dampening material or other device or configuration that permits the endplates and, thereby, the vertebral bodies to move relative to each other. The endplates need to be fixed to the vertebral bodies during initial insertion and over the life of the implant while the patient is moving their spine. Secure and relatively simple fixation features and methods are preferred to secure the endplate to the vertebral bodies.

It would be desirable to develop an implant that is able to be securely fixed to adjacent vertebral bodies utilizing a relatively simple surgical method.

### BRIEF SUMMARY OF THE INVENTION

The present invention relates generally to an intervertebral implant. More specifically, the present invention relates to an intervertebral implant including one or more spiral anchors for securing the implant between superior and inferior vertebral bodies.

The spinal implant preferably includes an intervertebral spacer and at least one spiral anchor securely coupled to both the intervertebral spacer and to at least one of the inferior and superior vertebral bodies, wherein the at least one spiral anchor is configured to be at least partially embedded within at least a portion of one of the inferior and superior vertebral bodies.

In one exemplary embodiment, the implant includes a plurality of spiral anchors which may be coupled to a single intervertebral spacer. Alternatively, one of the spiral anchors may be coupled to a superior spacer or plate for contacting the superior vertebral body while the other spiral anchor may be coupled to an inferior spacer or plate for contacting the inferior vertebral body. In this manner, the implant may be in the form of an articulating implant enabling movement between the superior spacer or plate and the inferior spacer or plate, and hence between the superior and inferior vertebral bodies.

In one preferred embodiment, an instrument for coupling a spiral anchor to an intervertebral spacer and embedding at least a portion of the spiral anchor into a vertebral body includes a cylindrical element having a proximal end, a distal end and a longitudinal axis extending therebetween. The distal end is configured to releasably mate with the spiral anchor and rotating the cylindrical element enables the spiral anchor to be coupled to the vertebral body and to embed within the vertebral body.

### BRIEF DESCRIPTION OF SEVERAL VIEWS OF THE DRAWINGS

The foregoing summary, as well as the following detailed description of preferred embodiments of the application, will be better understood when read in conjunction with the appended drawings. For the purposes of illustrating the preferred implants, there are shown in the drawings preferred embodiments. It should be understood, however, that the drawings are not intended to limit the scope of this invention, but merely to clarify and be illustrative of embodiments of the invention. In the drawings:

FIG. 1 illustrates a top perspective view of a spinal implant according to a first preferred embodiment of the present invention;

FIG. 2 illustrates a top perspective view of a spacer of the spinal implant of FIG. 1;

FIG. 3A illustrates a side perspective view of an exemplary instrument for implanting a preferred spiral anchor of the preferred spinal implants of the present invention;

FIG. 3B illustrates use of the instrument of FIG. 3A to insert the spiral anchor of FIG. 3A into a spinal implant adapted for insertion via an antero-lateral approach;

FIG. 4 illustrates a top perspective view of a spinal implant according to a second preferred embodiment of the present invention;

FIG. 5A illustrates a top perspective view of a spinal implant according to a third preferred embodiment of the present invention;

FIG. 5B illustrates a top perspective view of spiral anchors of the spinal implant of FIG. 5A;

FIG. 5C illustrates a top perspective view of a spinal implant according to a fourth preferred embodiment of the present invention;

FIG. 6 illustrates a top perspective view of a spinal implant according to a fifth preferred embodiment of the present invention; and

FIG. 7 illustrates a side, cross-sectional view of the spinal implant of FIG. 6, taken along line 7-7 of FIG. 6, wherein the spinal implant is shown inserted into an intervertebral disc space between adjacent vertebral bodies.

### DETAILED DESCRIPTION OF CERTAIN EMBODIMENTS

Certain terminology is used in the following description for convenience only and is not limiting. The words “right”, “left”, “top” and “bottom” designate directions in the drawings to which reference is made. The words “inwardly” and “outwardly” refer to directions toward and away from, respectively, the geometric center of the spinal implant and designated parts thereof. The words, “anterior”, “posterior”, “superior”, “inferior”, “lateral”, “sagittal”, “axial”, “coronal” and related words and/or phrases designate preferred positions and orientations in the human body to which reference is made and are not meant to be limiting. The terminology includes the above-listed words, derivatives thereof and words of similar import.

Certain embodiments of the present invention will now be discussed with reference to the aforementioned figures, wherein like reference numerals refer to like components. Preferred embodiments of the present invention are directed to an exemplary spinal implant with a spiral anchor or with multiple spinal anchors to secure the implant to adjacent vertebral bodies V.

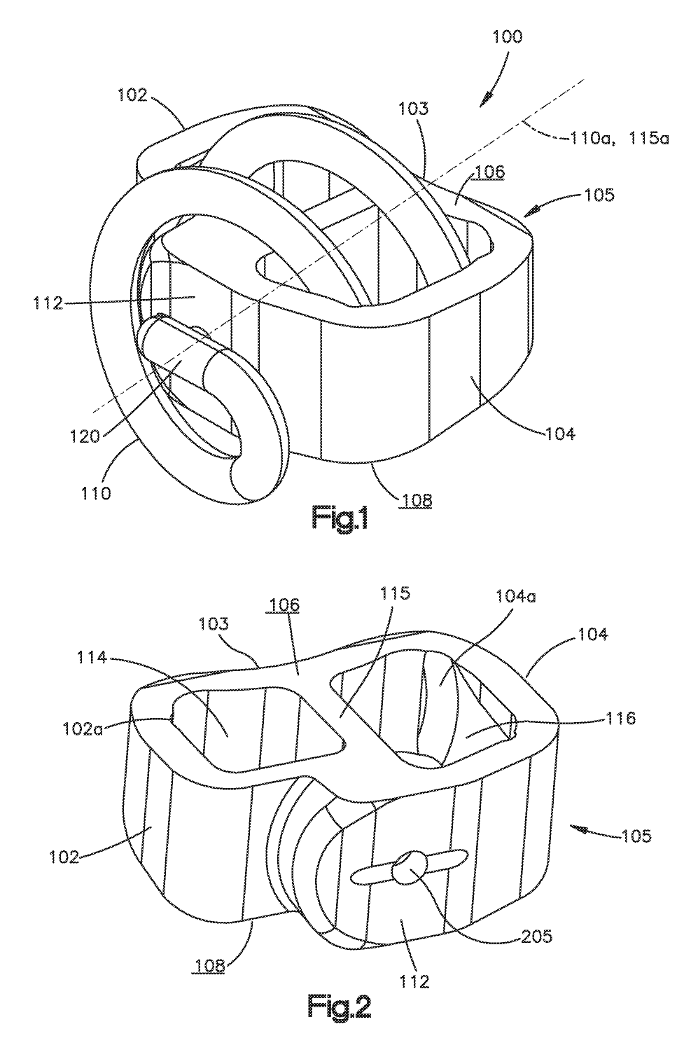

Referring to FIGS. 1-7, preferred embodiments of the present invention relate to a spinal implant 100, 500, 500′, 600 (“100-600”). It should be understood that while the various embodiments of the spinal implant 100-600 are generally described and illustrated in connection with a spinal fusion procedure, those skilled in the art will appreciate that the implant 100-600 as well as the components thereof may be used for non-fusion procedures, as will be described in greater detail below.

Generally, the various preferred embodiments of the spinal implant 100-600 are sized and configured for insertion into an intervertebral disc space D between adjacent vertebral bodies V. The implants 100-600 may be sized and configured to replace all or substantially all of an intervertebral disc space D between the adjacent vertebral bodies V or only part of the intervertebral disc space D. In addition, the preferred implants 100-600 may be configured to replace an entire vertebral body V and related disc spaces D in a patient's spine, as would be apparent to one having ordinary skill in the art, based upon a review of the present application. The spinal implant 100-600 may be adapted for use in the anterior, antero-lateral, direct lateral, extra-foraminal, transforaminal, and posterior approaches to the spine.

The preferred embodiments of the spinal implants 100-600 each preferably include an intervertebral spacer and one or more spiral anchors. Referring to FIG. 1, the spinal implant 100 of the first preferred embodiment includes an intervertebral spacer 105 and a spiral anchor 110. The implant 100 is preferably inserted via an anterior approach, but may also be adapted for insertion via an antero-lateral approach. As such, the spacer 105 may include a first lateral side 102, a second lateral side 104 that is opposite the first lateral side 102, an upper surface 106, a lower surface 108, a posterior end 103 and a protruding portion 112. The spacer 105 is preferably configured and dimensioned for implantation into the intervertebral disc space D between adjacent vertebral bodies V such that the upper and lower surfaces 106, 108 engage endplates of the vertebral bodies V and the spiral anchor 110 is engaged with the vertebral bodies V. The spacer 105 is sized and configured to maintain and/or restore a desired intervertebral disc height H between the adjacent vertebral bodies V. The spacer 105 of the first preferred embodiment includes at least two through holes 114, 116 that extend from the upper surface 106 through to the lower surface 108 and are separated by a separating wall 115. The through holes 114, 116 and separating wall 115 are configured to accommodate the spiral anchor 110 for securing the implant 100 to the adjacent vertebral bodies V. Preferably, one or both of the first and second lateral sides 102, 104 have concave-shaped grooves 102a, 104a on inner surfaces to accommodate the spiral anchor 110. Although the separating wall 115 is shown as being continuous between the upper surface 106 and the lower surface 108, the separating wall 115 may vary in size or height and/or be comprised of multiple pieces.

The spiral anchor 110 is preferably a generally helical shaped device that is mounted to the spacer 105 in an assembled configuration and includes an insertion end 118 that is to be inserted into a vertebral body V to anchor the implant 100 to an adjacent vertebral body V and a second end 120 which is secured to the spacer 105 in the assembled configuration. Alternatively, an intermediate portion between the insertion end 118 and the second end 120 of the spiral anchor 110 may be secured to the spacer 105 at a single or multiple sections along its length. As will be discussed in more detail below, multiple spiral anchors 110 may be used in conjunction with one or more spacers 105 in order to cover a wide range of surface area when coming in contact with the adjacent vertebral body V. Although described in terms of being generally helical, the spiral anchor 110 may assume a range of conventional structural geometries (e.g. triangular or trilobular, etc.) as is known in the art.

Both the spacer 105 and the anchor 110 may be manufactured from one or more bio-compatible materials known in the art, including, but not limited to, titanium, titanium alloys, Nitinol, stainless steel, PEEK, carbon fiber, impregnated PEEK, PDLA or PPLA, bioglass composites, allograft bones, etc. As will be appreciated by one of ordinary skill in the art, the implant 100 may also be coated with various compounds to increase bony on-growth or in-growth, promote healing, or allow for revision of the implant, including hydroxyapatite, titanium-nickel, vapor plasma spray deposition of titanium, or plasma treatment to make the surface hydrophilic.

The upper and lower surfaces 106, 108 of the spacer 105 of the first preferred embodiment may include a series of teeth, ridges, spikes, one or more keels, or other similar projections (not shown) to aid in securing the implant 100 to the endplates of the adjacent vertebral bodies V. The through holes 114, 116 may be packed with bone graft material to promote bone growth following implantation.

The upper and lower surfaces 106, 108 of the spacer 105 may be curved, parallel or tapered to adapt to the anatomy of a specific area of a patient's spine or the adapt to specific anatomical features of a patient's vertebral bodies V. The particular surface shape and curvature or taper in the anterior-posterior direction as well as between the first and second lateral sides 102, 104 of the upper and lower surfaces 106, 108 depends upon the location the implant 100 is intended to be implanted and/or surgeon preferences.

In the first preferred embodiment, the implant 100 is configured so that the spiral anchor 110 is coupled to the spacer 105 after the spacer 105 is implanted into the intervertebral disc space D as can be seen in FIG. 7. However, the spacer 105 and the spiral anchor 110 may be coupled together prior to being implanted into the disc space D.

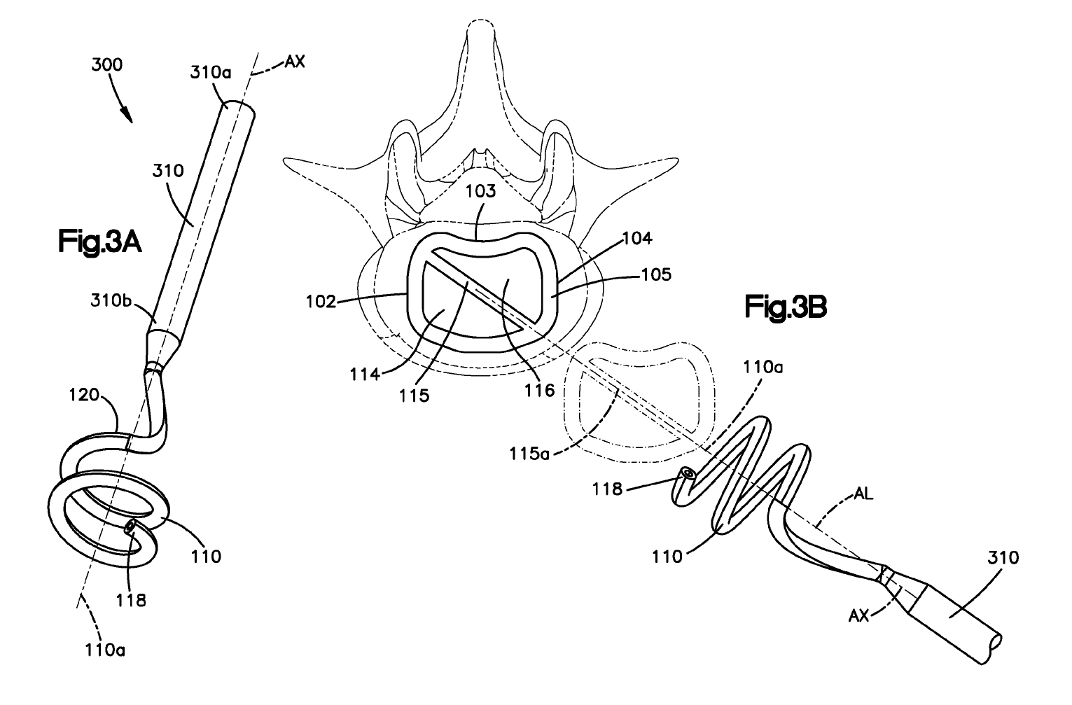

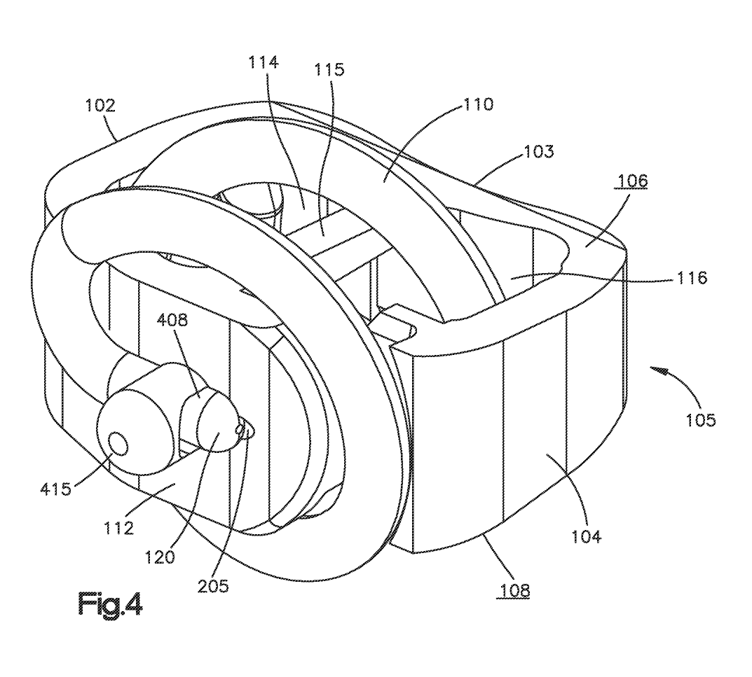

Referring to FIGS. 3A and 3B, an exemplary instrument 300 and procedure for inserting the implant 100 with a modified separating wall 115 into the intervertebral disc space D and for coupling the spiral anchor 110 to the spacer 105 and the adjacent vertebral bodies V will now be discussed, however, those skilled in the art will appreciate that the implant 100 may be inserted via any heretofore known or hereafter developed conventional surgical technique using any heretofore known or hereafter developed instruments. The instrument 300 for coupling the spiral anchor 110 to the spacer 105 and implantation into the adjacent vertebral bodies V includes a handle 310 having a proximal end 310a, a distal end 310b and a longitudinal axis AX. The distal end 310b of the instrument 300 is designed to releasably engage the second end 120 of the spiral anchor 110 for insertion into the spacer 105 and implantation into the adjacent vertebral bodies V. In the first preferred embodiment, the insertion of the anchor 110 into the spacer 105 occurs after the spacer 105 has been inserted into the disc space D. Alternatively, the insertion of the anchor 110 into the spacer 105 may occur prior to insertion of the spacer 105 into the disc space D. The instrument 300 may include a generally helical shaped insertion portion (not shown) wherein the helical shaped portion mates with the spiral anchor 110 to rotate the anchor 110 for insertion into the spacer 105 and into the adjacent vertebral bodies V.

After at least a portion of the disc space D is cleared out, a surgeon preferably inserts the posterior end 103 of the spacer 105 partially into the disc space D via a direct anterior approach. The surgeon then uses the instrument 300 to rotate the spiral anchor 110 through the spacer 105 such that the spiral anchor 110 engages the concave-shaped grooves 102a, 104a to urge the spacer 105 into the disc space D and embed the anchor 110 into the adjacent vertebral bodies V. Once the anchor 110 is embedded and the spacer 105 is positioned in a preferred position in the disc space D, the instrument 300 releases the anchor 110 and the anchor 110 may be secured to the spacer 105.

Referring to FIG. 3B, the spacer 105 may also be inserted into the disc space D via the antero-lateral approach. The spacer 105 is inserted such that the first lateral side 102 initially enters the disc space D and the surgeon manipulates the spacer 105 until the spacer 105 is centered on the endplate of the vertebral body V in an implanted position (FIG. 3B). The spacer 105 is preferably adapted and configured to receive the spiral anchor 110 along an antero-lateral insertion axis AL such that the longitudinal axis AX of the instrument 300 is generally coaxial with the antero-lateral insertion axis AL during insertion of the spiral anchor 110. The spacer 105 preferably includes features (not shown), similar to the concave-shaped grooves 102a, 104a, to guide insertion and properly position the spiral anchor 110 relative to the spacer 105. The spacer 105 also preferably includes a modified separating wall 115 that is oriented generally parallel to the antero-lateral insertion axis AL in the implanted position and may be inserted with the modified separating wall 115 oriented generally parallel to the antero-lateral insertion axis AL. In the assembled configuration and the implanted position for the direct anterior approach (FIG. 1), the longitudinal axis 115A of the separating wall 115 and the longitudinal axis 110A of the spiral anchor 110A are preferably positioned parallel or coaxial.

Referring to FIG. 2, the protruding portion 112 may include an indentation or a concave surface 205 on its outer wall that mates with the second end 120 of the spiral anchor 110 and secures the position of the spiral anchor 110 relative to the spacer 105 once the spiral anchor 110 is located in the assembled configuration. In the first preferred embodiment, the indentation 205 on the outer wall of the protruding position 112 allows the spiral anchor 110 to be securely coupled with respect to the spacer 105 at only one position per every full rotation (e.g., 360°), such that a surgeon generally knows at which point the spacer 105 and the spiral anchor 110 are implanted optimally with respect to the disc space D. Accordingly, the second end 120 of the spiral anchor 110 will only mate with the indentation 205 once it has been fully rotated and is properly located in the assembled configuration relative to the spacer 105. Thus, the surgeon will know a full rotation has occurred when the second end 120 returns to its original position and mates with the indentation 205. Alternatively, the spiral anchor 110 may be coupled to the spacer 105 by any mechanism now known or hereafter developed for such purpose.

Referring to FIG. 4, in a second preferred embodiment, the spiral anchor 110 includes a driver tang 408 that can be rotated relative to the spacer 105 about a pin 415 to rotate the spiral anchor 110 through the spacer 105 and insert the anchor 110 into the adjacent vertebral bodies V using a pliers-type or other driver tool. In this second preferred embodiment, the outer wall of the protruding portion 112 may also contain a concave surface or indentation 205 for mating with the second end 120 of the anchor 110. Preferably, if there is one indentation 205 one or two full rotations of the driver tang 408 may indicate that the spacer 105 and the spiral anchor 110 have been optimally inserted with respect to the disc space D. The pin 415 is preferably, securely coupled to the spiral anchor 110 and reduces the possibility of the spiral anchor 110 backing out of the spacer 105 or the adjacent vertebral bodies V. Alternatively, a number of alternate locking mechanisms (not shown) can be provided to assure the surgeon that the spacer 105 and the spiral anchor 110 are optimally positioned with respect to the adjacent vertebral bodies V, such as a stop mechanism that interfaces with the spiral anchor 110 or drive tang 408. In the second preferred embodiment, the anchor 110 is pre-assembled to the spacer 105 and the anchor 110 and spacer 105 are urged as a single unit into the disc space D.

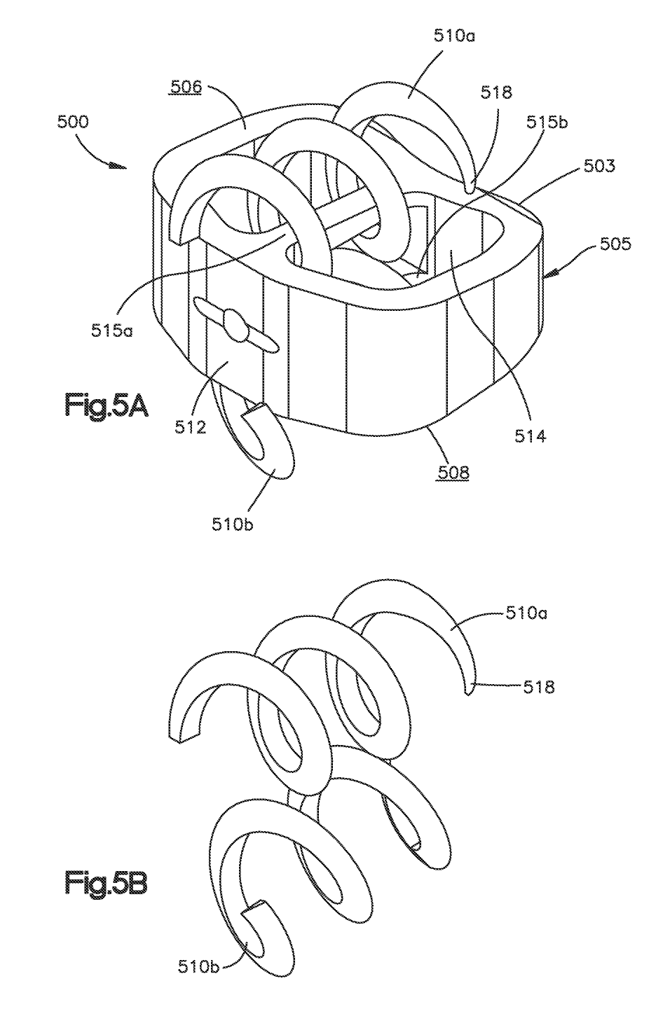

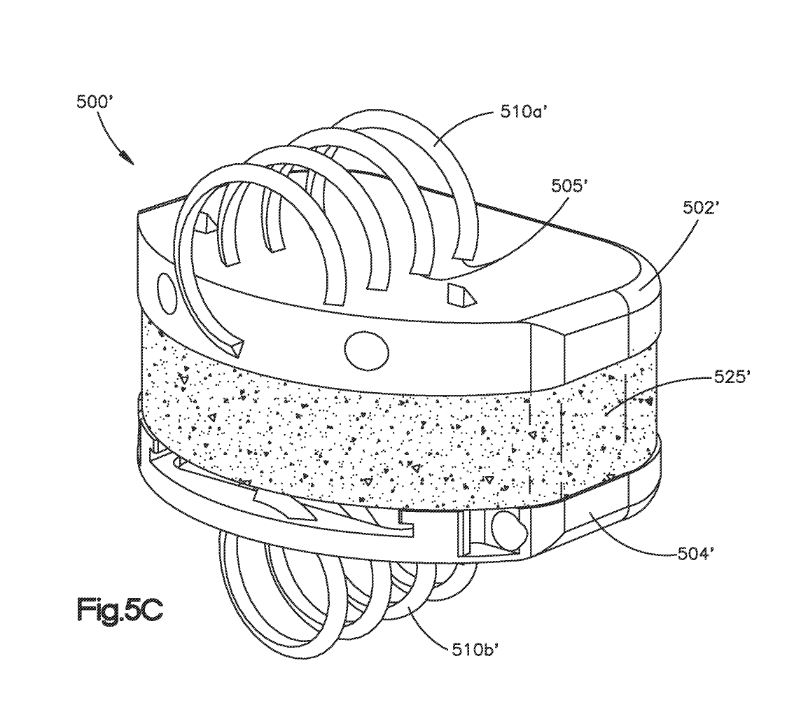

Referring to FIGS. 5A-5C, in a third preferred embodiment, the implant 500 includes two spiral anchors 510a, 510b that are configured to engage with the spacer 505. Each of the pair of anchors 510a, 510b includes an insertion end 518, preferably having a pointed or bullet-nosed tip that is initially inserted into the adjacent vertebral bodies V to ease insertion into the bone. The spacer 505 preferably includes an upper crossbar 515a and a lower crossbar 515b that extend across the spacer 500 from the posterior end 503 to an anterior end 512. The upper crossbar 515a extends along the upper surface 506 of the spacer 505 and is configured to accommodate the upper anchor 510a while the lower crossbar 515b extends along the lower surface 508 of the spacer 505 and is configured to accommodate the lower anchor 510b. By incorporating the upper and lower spiral anchors 510a, 510b, the user may engage each adjacent vertebral body V separately. In addition, the upper spiral anchor 510a may be coupled to a superior portion of the spacer 505 or to a superior endplate 502′ of a dynamic implant 500′ for contacting and engaging the superior vertebral body V while the lower spiral anchor 510b may be coupled to an inferior portion of the spacer 505 or to an inferior endplate 504′ of the dynamic implant 500′ for contacting and engaging the inferior vertebral body V. In this manner, the dynamic implant 500′ may be in the form of an articulating implant enabling movement between the upper endplate 502′ and the lower endplate 504′, and hence between the superior and inferior vertebral bodies V.

Referring specifically to FIG. 5C, the upper spiral anchor 510a′ secures the superior endplate 502′ to the superior vertebral body and the inferior spiral anchor 510b′ secures the inferior endplate 504′ to the inferior vertebral body V. The dynamic implant 500′ also includes a central element 525′ for enabling the superior endplate 502′ to move with respect to the inferior endplate 504′. The central element 525′ may be any motion enabling element now or hereafter known in the art including, for example, an elastomeric core, one or more articulating elements, a fluid filled bellows, etc. The superior and inferior spiral anchors 510a′, 510b′ may be coupled to the superior and inferior endplates 502′, 504′ by any mechanism herein described or now or hereafter known in the art. For example, the superior and inferior endplates 502′, 504′ may include a plurality of perforation 505′ for receiving the spiral anchors 510a′, 510b′ therein and for guiding the spiral anchor 510a′, 510b′ into the implanted position to maintain a preferred orientation and position of the spiral anchors 510a′, 510b′ relative to the superior and inferior endplates 502′, 504′.

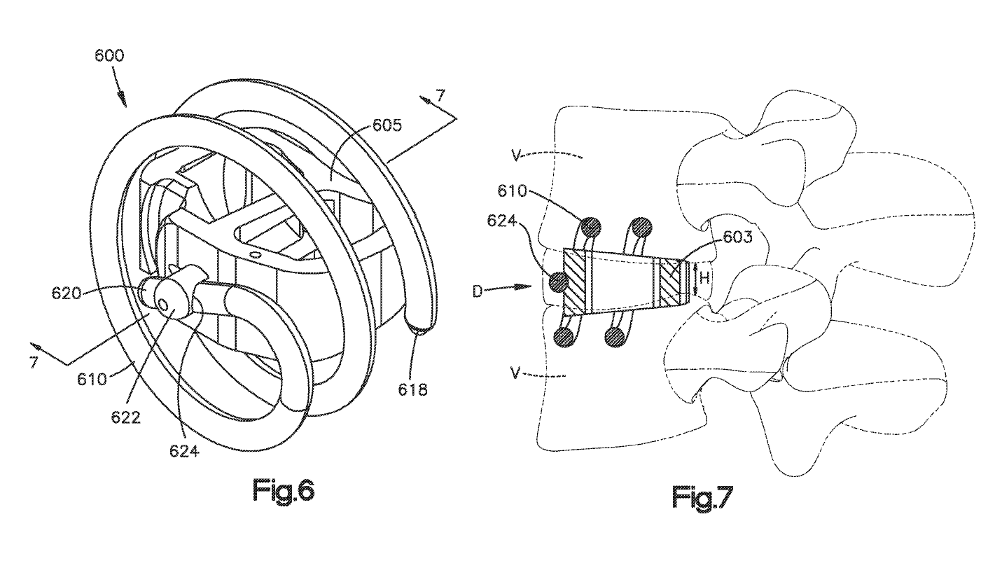

Referring to FIGS. 6 and 7, in a fourth preferred embodiment, the implant 600 preferably includes spiral anchor 610 which may be coupled to the spacer 605 at a point on the outer surface of the spacer 605. In this fourth preferred embodiment, the spacer 605 contains a member 622 that projects from one of the outer walls of the spacer 605. The member 622 preferably includes a bore 624 that is configured to receive the second end 620 of the anchor 610. The insertion of the second end 620 into the bore 624 preferably rigidly couples the anchor 610 to the spacer 605 so that the insertion end 618 can be inserted into a vertebral body V to anchor the implant 600 to the adjacent vertebral bodies V. The spiral anchor 610 may extend around the spacer 605 such that the anchor 610 does not contact the spacer 605 at any point other than being received within the bore 624.

Those skilled in the art will recognize that the method and system of the present invention has many applications, may be implemented in many manners and, as such, is not to be limited by the foregoing embodiments and examples. Any number of the features of the different embodiments described herein may be combined into one single embodiment and alternate embodiments having fewer than or more than all of the features herein described are possible. Functionality may also be, in whole or in part, distributed among multiple components, in manners now known or to become known. Moreover, the scope of the present invention covers conventionally known and features of those variations and modifications through the components described herein as would be understood by those skilled in the art. It is the intention, therefore, to be limited only as indicated by the scope of the claims appended hereto.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A spinal implant for insertion into an intervertebral disc space between first and second vertebral bodies, the implant comprising:
an intervertebral spacer; and
a spiral anchor securely coupled to both the intervertebral spacer and to the first and second vertebral bodies, the spiral anchor configured to at least partially embed within at least a portion of the first and second vertebral bodies, a longitudinal axis of the spinal anchor being generally parallel to a longitudinal axis of the spacer in an assembled configuration.

2. The implant of claim 1, wherein the spacer includes concave-shaped grooves, the spiral anchor positioned within the concave-shaped grooved in the assembled configuration.

3. The implant of claim 1, wherein the spiral anchor is comprised of a first spiral anchor and a second spiral anchor, the first spiral anchor is coupled to both the intervertebral spacer and the first vertebral body and the second spiral anchor is coupled to both the intervertebral spacer and to the second vertebral body in an implanted position.

4. The implant of claim 1, wherein the intervertebral spacer comprises an upper surface, a lower surface, a pair of through holes extending from the upper surface to the lower surface and a separating wall separating the pair of through holes, the spiral anchor is configured to be at least partially received within the through holes in the assembled configuration.

5. The implant of claim 1, wherein the spacer includes a protruding portion.

6. The implant of claim 5, wherein the protruding portion includes a bore, the bore positioned proximate a second end of the anchor in the assembled configuration.

7. The implant of claim 6, wherein the spiral anchor extends around the intervertebral spacer such that the anchor does not contact the spacer at any point other than being received within the bore in the assembled configuration.

8. The implant of claim 1 further comprising:
a rotatable pin that couples the spiral anchor to the intervertebral spacer so that the spiral anchor and pin can be rotated to facilitate insertion of the anchor into the vertebral bodies.

9. The implant of claim 1, wherein the spiral anchor includes a driver tang so that rotation of the driver tang enables the embedding of the spiral anchor.

10. A method for inserting an implant including a spacer portion and a spiral anchor into an intervertebral disc space between first and second vertebral bodies, the method comprising the steps of:
a) inserting the spacer portion of the implant into the disc space;
b) coupling an instrument to the spiral anchor;
c) rotating the spiral anchor into engagement with the intervertebral spacer and into engagement with at least one of the first and second vertebral bodies, rotation of the at least one spiral anchor at least partially embedding the anchor within at least a portion of one of the first and second vertebral bodies; and
d) releasing the spiral anchor from the instrument.

11. The method of claim 10, wherein rotation of the spiral anchor in step (c) at least partially embeds the anchor within the first and second vertebral bodies.

12. The method of claim 11, wherein step (c) includes rotating a first spiral anchor into engagement with the intervertebral spacer and into engagement with the first vertebral body and rotating a second spiral anchor into engagement with the intervertebral spacer and into engagement with the second vertebral body.

13. A spinal implant for insertion into an intervertebral disc space between first and second vertebral bodies, the implant comprising:
a superior endplate adapted for engaging the first vertebral body;
an inferior endplate adapted for engaging the second vertebral body;
a superior spinal anchor secured to the superior endplate and the first vertebral body in an implanted position; and
an inferior spiral anchor secured to the inferior endplate and the second vertebral body in the implanted position.

14. The spinal implant of claim 13 further comprising:
a central element mounted between the superior and inferior endplates, the central element permitting motion between the superior and inferior endplates.

15. The spinal implant of claim 14 wherein the central element is comprised of an elastomeric core.

16. The spinal implant of claim 14 wherein the central element is comprised of an articulating element.

17. The spinal implant of claim 14 wherein the central element is comprised of a fluid filled bellows.

18. The spinal implant of claim 13 wherein the superior and inferior endplates include a plurality of perforations to accommodate the superior and inferior spiral anchors, respectively, in an assembled configuration.
